(19) United States
(12) Patent Application Publication
(10) Pub. No.: US 2009/0093786 A1
(43) Pub. Date: Apr. 9, 2009

(54) METHOD FOR DETECTING THE OCCLUSION OF A TUBING FOR A DEVICE FOR ADMINISTERING PHYSIOLOGICAL LIQUIDS

(76) Inventors: Serge Renaux, Les Aires (FR); Raymond Louberssac, Saint-Raphael (FR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/093,597

(22) PCT Filed: Nov. 7, 2006
(86) PCT No.: PCT/FR2006/002488

§ 371 Date: Oct. 6, 2008

(30) Foreign Application Priority Data
Nov. 16, 2005 (FR) 0511610
Nov. 6, 2006 (FR) 0609671

(51) Int. Cl. A61M 5/142

(52) U.S. Cl. 604/500; 604/67

(57) ABSTRACT

The invention relates to a self-parameterizable method for detecting occlusion and pulling out of a tubing for ad-ministering, by enteral route, physiological liquids and to a device for carrying out said method. The inventive device comprises, in essence: a pump (1); an administering tubing (2, 3); a tri-route tubular structure (4, 5, 7) provided with an occlusion detection area (7); a computer (14) and an electromagnetic detection means (8, 9) for detecting the presence of a fluid in said detection area (7) and for detecting a pulling out of said tri-route structure; an electromagnetic detection means (15, 16) for detecting the presence of air inside the dispensing tubing and the pulling out thereof. The inventive method is characterized, in essence, in that it consists of, in the event of repetitive occlusions, programming the computer (14) for systematically and automatically calculating the intensity of the wave received by the receiver (9) at the time of each occlusion and using this value as a reference value for determining the new detection intensity threshold to use during each starting or restarting.

### FIELD OF THE INVENTION

The present invention relates to a method for detecting occlusion and pulling out of a tubing designed for the administration, by enteral pathway, of physiological liquids, as well as the apparatus designed for the implementation of the aforementioned method.

### TECHNICAL BACKGROUND

Equipment for administration, via enteral pathway, of physiological liquids, is known, this equipment being of the type including:

a) a pump, particularly the linear or rotary peristaltic type;

b) a tubing designed for the administration of the aforesaid liquids;

c) an occlusion detection device, comprised:
of a fluid inlet zone coupled to a first part of the aforementioned administration tubing and a fluid outlet zone coupled to a second part of the aforementioned tubing;
of an occlusion detection zone, arranged along a direction substantially perpendicular to the flow direction of the aforementioned fluid, being part of a tri-route tubular structure, arranged so that the aforementioned fluid enters the aforementioned detection zone in case of occlusion of the aforementioned second part of the aforementioned tubing;
of a means of detection of the presence of a fluid in the aforementioned detection zone, comprised of an emitter of an electromagnetic wave and a receiver of the aforementioned electromagnetic wave;

d) a computer programmed to measure the intensity the electromagnetic wave received at the aforementioned receiver in order to detect the presence of the aforementioned fluid in the aforementioned detection zone when the intensity of the electromagnetic wave reaches a predefined threshold and trigger, in the latter case, an alarm.

Such devices are essentially described in patents FR2945475 and GB6183437.

They all present principal disadvantages related to:

a) the detection of occlusion after the fluid enters the detection zone after a first occlusion: in fact, the characteristics of radiation received at the means of detection are disrupted since the sensing zone was moistened by the fluid, causing a change in surface tension due to traces that can stagnate in front of the means of detection, requiring the changing of the tube at each occlusion, which is not practical;

b) the pulling out of the tri-route tubular structure as well as the second part of the administration tubing, which has the effect of rendering the pump inoperable;

c) the lack of adjustment means, other than to move the receiver and emitter assembly, to vary the threshold for triggering the alarm.

### SUMMARY OF INVENTION

The invention is directed toward developing a method and realizing a device for its implementation that eliminates the disadvantages mentioned above.

To accomplish this, the method according to the invention is essentially characterized in that it comprises:
in the case of repetitive occlusions, programming the computer to automatically and systematically measure the intensity of the wave received by the receiver at the time of each occlusion and using this value as a reference value to determine the new detection intensity threshold to use at each start-up or reboot, particularly for taking take into account the traces of liquid that could stagnate and thus avoid having to change the tri-route tubular structure at each occlusion detection;
in order to adapt to the type of liquid in the aforementioned tubing, programming the computer to measure, for a well-defined time, the value of the voltage between the terminals of the means of detection and establishing this value as a reference value until the next change of the aforementioned tubing;
in order to change the threshold for triggering the alarm, varying the volume of compressible air contained in the occlusion detection zone;
in order to detect the pulling out of the tri-route tubular structure as well as the presence of a fluid in its detection zone, using a means of electromagnetic detection, particularly the infrared type;
in order to detect pulling out of the second part of the administration tubing as well as the presence of air in it, using a means of electromagnetic detection, particularly the ultrasound type.

To accomplish this, the device designed for the implementation of the aforementioned method is characterized essentially in that it includes
a pump, an administration tubing, a tri-route tubular structure having an occlusion zone detection, a computer;
an electromagnetic means of detecting pulling out of the aforementioned tri-route tubular structure and of the presence of a fluid in aforementioned occlusion detection zone;
an electromagnetic means of detecting pulling out of the second part of distribution tubing and of the presence of air in it;
a cap, having adjustable height, or being interchangeable, adapted to vary the volume of air in the occlusion detection zone and thus the trigger threshold of the alarm.

In the absence of occlusion, the fluid pressure is such that it passes through the tubing without entering the detection zone.

In case of occlusion of the tubing, for example if the patient pliers or twists it or coughs strongly but in this case there is no mechanical occlusion but temporary reflux during the fit of coughing or if the probe is clamped or blocked, the pressure will increase within the tubing, and in response to this increased pressure, the fluid will enter the detection zone. When the meniscus of the liquid passes, for example, the axis of the detection threshold, the electromagnetic receiver will record a change of the received wave enabling the detection of the occlusion.

The detection zone is in this case substantially transparent to the aforementioned electromagnetic radiation, the aforementioned fluid being substantially opaque to the aforementioned radiation, so that the occlusion detection can be carried out by measuring the intensity at the aforementioned receiver.

Preferably, the aforementioned means of detection defines a detection axis, the aforementioned detection zone traversing the aforementioned detection axis. In this way, the fluid can be detected when it crosses the detection axis.

The aforementioned detection zone can be arranged along a direction substantially perpendicular to the flow direction of the aforementioned fluid between the aforementioned fluid inlet zone and the aforementioned fluid outlet zone. In this way, the detecting zone can easily be positioned along a detection axis at the means of connection. To ensure that the measures of radiation intensity can be accurate even if the detection zone has already been crossed by fluid, the method can also include a calibration step of measuring the intensity of the aforementioned electromagnetic radiation received at the aforementioned receiver so as to define a calibration value of the aforementioned intensity, the aforementioned predefined threshold depending on the aforementioned calibration value of the aforementioned intensity.

In normal operation, the pressure in the air stopper is stronger than the pressure inside the fluid inlet and outlet zones near the occlusion detection zone and the meniscus fluid does not reach the detection axis. The electromagnetic radiation emitted by the emitter is received by the receiver after crossing the detection zone containing the air.

In case of occlusion of the downstream section of the tubing, the pressure at the detection zone increases and the meniscus of fluid enters into the aforementioned zone beyond the detection axis by compressing the air situated in it. In this case, the radiation received by the receiver is changed.

The emitter and receiver are, for example, coupled to a computer, possibly integrated with the pump, which calculates the radiation intensity received by the receiver by processing the received signal.

The computer is programmed to then emit an alarm in case of change of radiation received at the receiver.

It will be noted that the programming of the computer depends on the characteristics of the fluid distributed and on the type of detection used. According to a mode of implementation, for example, nutrients that are opaque to the chosen electromagnetic radiation are distributed and a transceiver-receiver pair adapted to emit and detect the aforementioned radiation is used. In this case, when the meniscus of fluid penetrates to the detection axis of the detection zone, the signal emitted by the emitter is blocked by the nutrients and is no longer received by the receiver. The computer then detects this change of the received signal and triggers the alarm.

### PRESENTATION OF THE FIGURES

The characteristics and advantages of the invention will appear more clearly upon reading the following detailed description of a preferred mode of implementing it given by way of non-limiting example and represented in the attached drawings.

Of these drawings:

FIG. 1 is a schematic of the assembly of the apparatus according to the invention;

FIG. 2 is a detail section view of the tri-route tubular structure and of the cap sealing and delimiting the volume of compressible air of the occlusion detection zone;

FIGS. 3 and 4 are respectively top and front views of the occlusion detection device emphasizing the positioning fins;

FIG. 5 is a profile view of the occlusion detection device emphasizing the square shape of the occlusion zone and its position between the forks of electromagnetic radiation.

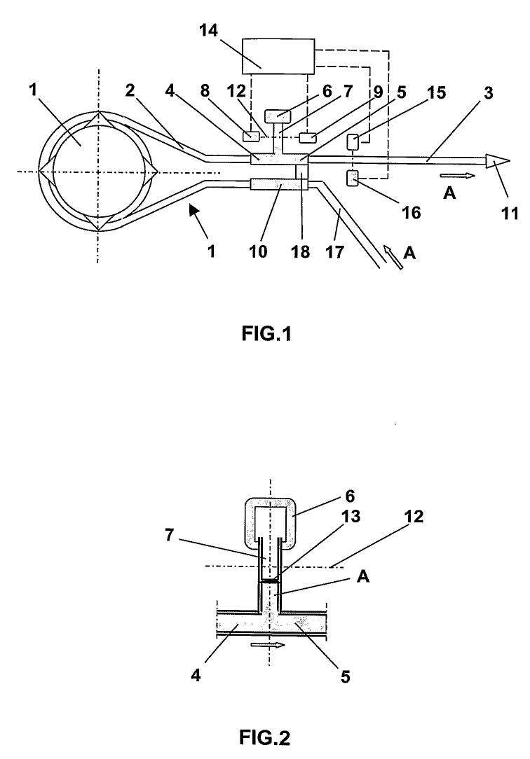

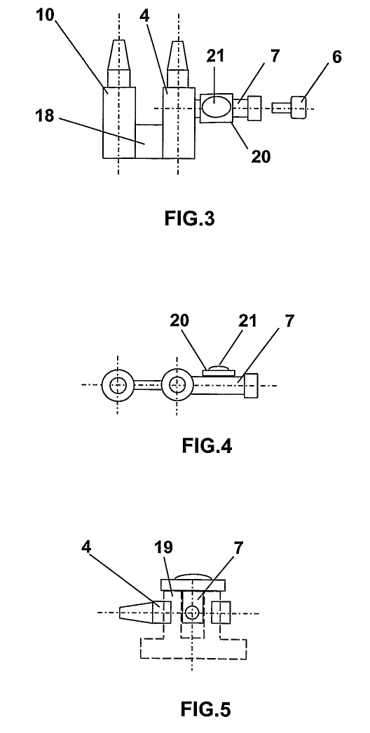

### DETAILED DESCRIPTION OF INVENTION

The apparatus represented in the figures, designed for the administration, by enteral pathway, of physiological liquids (A), particularly nutritional, medical or hybrids, essentially includes:

a) a pump (1), or a pumping system, particularly the linear or rotary peristaltic type, fixed rate or programmable;

b) an administration tubing (2, 3);

c) an occlusion detection device (4, 5, 7) comprised:
of a fluid inlet zone (4) coupled to a first part (2) of the administration tubing and a fluid outlet zone (5) coupled to a second part (3) of the aforementioned tubing;
of a zone (7), designed for the detection of an occlusion, arranged along a direction substantially orthogonal to the direction of flow of the aforementioned fluid, forming a tri-route tubular structure (4, 5, 7), arranged so the aforementioned fluid enters into the aforementioned detection zone (7) in case of occlusion of the second part (3) of the aforementioned administration tubing (2, 3);
of a means of detection (8, 9) of the presence of a fluid (A) in the aforementioned detection zone (7), comprising an emitter (8) of an electromagnetic wave and a receiver (9) of the aforementioned electromagnetic wave defining a detection axis (12); the aforementioned fluid being substantially opaque to the electromagnetic wave;

d) a computer (14) programmed to measure the intensity of the aforementioned electromagnetic wave received at the aforementioned receiver in order to detect the presence of the aforementioned fluid in the detection zone when the intensity of the electromagnetic wave reaches a predefined threshold and trigger, in the latter case, an alarm.

The self-parameterizable method of detecting occlusion and pulling out of the tubing (2, 3) comprises:
in the case of repetitive occlusions, programming the computer (14) to systematically and automatically measure the intensity of the wave received by the receiver (9) at the time of each occlusion and using this new value as a reference value for determining the new threshold of detection intensity to use at each start-up or reboot;
in order to adapt to the type of liquid present in the tri-route tubular structure (4, 5, 7), programming the computer (14) to measure, for a predetermined time, the value of the voltage between the terminals of the means of detection and establishing this value as a reference value until the next change of the aforementioned tubular structure;
in order to change the threshold for triggering the alarm, varying the volume of compressible air contained in the occlusion detection zone (7);
using the means of electromagnetic detection (8, 9), particularly the infrared type, to also detect the presence in its fixation zone, of the tri-route tubular structure (4, 5, 7);
in order to detect the presence, in its fixation zone, of the second part (3) of the administration tubing (2, 3) and the presence of air in it, using a means (15, 16), comprising an emitter (15) of an electromagnetic wave, particularly the ultrasound type, and a receiver (16) of the aforementioned electromagnetic wave;
programming the computer (14) to not trigger the alarm until after a predetermined time in relation to the detection of the trigger threshold.

The represented apparatus also includes:
intake tubing (17) leading from the reservoir containing the liquid to be administered;
a sleeve (10) enabling coupling of the aforementioned tubing at the end of the tubing (2) that passes in the pump (1);
a probe connector (11) designed for enteral administration;
in the upper occlusion detection zone (7), an end cap (6), with adjustable height (in particular, by screwing), or interchangeable, adapted to vary the volume of air in the aforementioned occlusion detection zone;
a wall (18) adapted to couple the tri-route tubular structure (4, 5, 7) to the sleeve (10) in order to form a single piece.

The meniscus of the liquid column in the occlusion detection zone (7), bears the reference mark (13).

The detection device including the detection zone (7), the fluid inlet zone (4), and the fluid outlet zone (5), may be connected to a tubing, or may be composed of one piece within the tubing itself.

It includes means of connecting, at the inlet and outlet zones, adapted to the tubing used and known in the medical field.

Tubing used in association with the occlusion detection device are, for example, PVC tubes, with or without phthalate, or tubes based on styrenic material of the SEBS type or silicone.

The occlusion detection device can be made of flexible or rigid material, but a rigid molded material is preferred.

The type of material used for the occlusion detection device 1 is for example a rigid polymer of the ABS type, or of the 30 Terlux type (registered trademark) designating a material of the MABS (Methylmethacrylate-Acrylonitrile-Butadiene-Styrene) type. The material used for the occlusion detection device is also chosen so that it can be glued to PVC tubings.

The person of skill in the art will also understand that the means of detection (8) and (9) can be positioned at the distribution pump (1) to achieve a compact device. In this case, the computer (14) may be the computer coupled to the aforementioned pump. It is also possible to use means of detection separated from the pump and having a dedicated computer.

The means of detection may also serve to fix the detection zone (7) so that it does not leave the radiation zone. Various means of fixing the means of detection may also be considered in order to clamp the detection zone (7) between the emitter part (8) and the receiver part (9). In particular, the occlusion detection zone (7) may have a square shape designed to fit perfectly between the forks (19) supporting the emitter (8) and receiver (9) of the electromagnetic wave and include two wings (20) adapted to being positioned in support on the aforementioned emitter and receiver in order to ensure its correct placement on the path of the aforementioned wave and a means (21) having an effect of a magnifying glass adapted to visually verify that the alarm generated by the computer (14) was caused by an occlusion, pulling out, or improper positioning of the tri-route tubing (4, 5, 7). Finally, it is noted that the invention can be used with any type of fluid traversing a flexible tubing for which an occlusion is possible.

The computer data can be stored locally to be used in real-time or deferred or be transmitted at a distance to a central treatment element. In this case, the local alarms can be inhibited.

We have described the present invention in the context of a medical use for which an occlusion may have very negative consequences, but it is understood that the present invention depends neither on the fluid circulated in the flexible tubing, nor the type of detection used.

The person of skill in the art is indeed capable of adapting the means of detection depending on the circulated fluid and will be able to choose the emitters and the receivers throughout the electromagnetic spectrum, depending on the fluid and constraints of field of application.

1-11. (canceled)

12. A method for detection of occlusion and pulling out of a tubing of an apparatus for the administration, by enteral pathway, of physiological liquids, the apparatus including:
a) a pump;
b) an occlusion detection device including:
a fluid inlet zone coupled to a first part of the tubing and a fluid outlet zone coupled to a second part of the tubing;
a zone, configured for the detection of an occlusion, arranged along a direction substantially perpendicular to the direction of flow of the fluid, forming a tri-route tubular structure, arranged so that the fluid enters the detection zone in case of occlusion of the second part of the tubing;
a detector configured to detect the presence of a fluid in the detection zone, the detector including an emitter of an electromagnetic wave and a receiver of the electromagnetic wave; and
a computer programmed to measure the intensity of the electromagnetic wave received at the receiver in order to detect the presence of the fluid in the detection zone when the intensity of the electromagnetic wave reaches a predefined threshold and trigger an alarm,
the method comprising
programming the computer to automatically measure an intensity of the wave received by the receiver at the time of each occlusion; and
using the measured intensity as a reference value for determining a new threshold of intensity detection to use at each start-up or reboot.

13. The method according to claim 12 further including, in order to adapt to the type of liquid present in the tri-route tubular structure, programming the computer to measure, for a predetermined time, the value of voltage between terminals of the detector and establishing this value as a reference value until the next change of the tubular structure.

14. The method according to claim 12 further including, in order to change the threshold for triggering the alarm, varying the volume of compressible air contained in the occlusion detection zone.

15. The method according to claim 12 further including using the detector to also detect the presence of the tri-route tubular structure.

16. The method according to claim 12 further including, in order to detect the presence of the second part of the administration tubing and the presence of air in it, using an emitter of an electromagnetic wave and a receiver of the electromagnetic wave.

17. The method according to claim 15 further including using an infrared wave as an electromagnetic wave.

18. The method according to claim 12 further including, in order to detect the presence of the second part of the administration tubing and the presence of air in it, using an emitter of an ultrasound wave and a receiver of the ultrasound wave.

19. An apparatus for the implementation of the method according to claim 1, wherein it includes a pump, an administration tubing, a tri-route tubular structure provided with an occlusion detection zone, a computer and a means of detection, comprised of an emitter of an electromagnetic wave and a receiver of the electromagnetic wave, adapted to detect the presence of a fluid in the zone for detection of occlusion and the pulling out of the tri-route tubular structure;
a means, comprised of an emitter of an electromagnetic wave and a receiver of the electromagnetic wave, adapted to detect the presence of air in the second part of the administration tubing and the pulling out of it; and
an end cap, with adjustable height, or interchangeable, adapted to vary the volume of air in the occlusion detection zone.

20. The method according to claim 19 wherein the occlusion detection zone has a square shape adapted to fit between forks supporting the emitter and the receiver of the electromagnetic wave, and including two wings adapted to being positioned in support on the aforementioned emitter and receiver in order to ensure its correct placement on the path of the aforementioned wave and a magnifier to enable visual verification that the alarm generated by the computer was caused by an occlusion, pulling out, or improper positioning of the tri-route tubular structure.
